(19) United States
(12) Patent Application Publication
(10) Pub. No.: US 2026/0013744 A1
(43) Pub. Date: Jan. 15, 2026

(54) SYSTEMS AND METHODS OF DETECTING USER VITALS BASED ON VIDEO STREAM DATA

(71) Applicant: AIZtech Canada Inc., Toronto (CA)

(72) Inventors: Azhar Quddus, Calgary (CA); Lingxiao Qi, Toronto (CA); Hassan Mahmoud Mohamed Ramadan Mahamed, Mississauga (CA); Mahmut Karakaya, Alpharetta, GA (US); Benjamin Fonooni, Toronto, CA (US); Mohamed Sheta, Toronto (CA); Andrew Franklin Arthur, Hollis, NH (US)

(21) Appl. No.: 19/244,713

(22) Filed: Jun. 20, 2025

Related U.S. Application Data
(60) Provisional application No. 63/670,525, filed on Jul. 12, 2024.

(51) Int. Cl. A61B 5/024; G06T 7/00; G16H 50/30

(52) U.S. Cl. CPC A61B 5/02416; G06T 7/0016; G16H 50/30

(57) ABSTRACT

Systems and methods of detecting user vitals based on video stream data. The system includes a processor and a memory. The memory may store processor-executable instructions that, when executed, configure the processor to receive an image data set representing a user face over an evaluation period; generate a remote photoplethysmogram (PPG) signal based on the image data set; determine a recovered PPG signal by generating a frequency response based on a frequency-tuned filter bank and the remote PPG signal, the recovered PPG signal generated based on a peak wavelet magnitude of the frequency response; generate a predicted electrocardiogram (ECG) signal based on a prediction model where one or more prediction model decoders tuned based on semantic features of the prior identified remote PPG signal; and determine, for display at a user device, user vitals data associated with the evaluation period.

### CROSS REFERENCE TO RELATED APPLICATIONS

This application claims priority from U.S. provisional patent application No. 63/670,525, entitled “SYSTEMS AND METHODS OF DETECTING USER VITALS BASED ON VIDEO STREAM DATA”, filed on Jul. 12, 2024, the entire contents of which are hereby incorporated by reference herein.

### FIELD

Embodiments of the present disclosure generally relate to health monitoring systems and devices and to systems, devices, and methods of detecting user vitals data.

### BACKGROUND

Heath monitoring devices may be configured for monitoring user activity. For example, such devices may include operations for tracking user fitness data and measuring a user's physiological metrics, such as heart rate variability, glucose measures, blood pressure reading, or other health-related information. Such health monitoring devices may be donned by a user on various portions of a user's body.

### SUMMARY

Features of embodiments of systems, devices, and methods for detecting user vitals will be described in the present disclosure.

In some embodiments, systems and devices may be configured to generate remote photoplethysmogram (PPG) signals based on video stream data. Remote PPG signals can be derived by a variety of methodologies. In some embodiments, plane-orthogonal-to-skin (POS) [12] methodology may be adapted. Thereby generate predicted electrocardiogram (ECG) signals based on the remote PPG signals. The video stream data may represent a time-series collection of images representing a user's face. Such example implementations may enable detection of user vitals based on non-contact interactions with a user's body.

In some embodiments, systems and devices may be configured to generate a plurality of ECG signals. Respective ECG signals may represent predicted data signals generated as if the ECG signals were acquired via a plurality of bioelectrode devices positioned across the user's body. As examples, simulated bioelectrode devices may include inferior ECG leads, lateral ECG leads, septal ECG leads, or anterior ECG leads.

Further, example implementation details of systems, devices, and methods for detecting user vitals will be described in the present disclosure.

In one aspect, the present disclosure describes a system for detecting user vitals. The system includes: a processor and a memory coupled to the processor. The memory may store processor-executable instructions that, when executed, configure the processor to: receive an image data set representing a user face over an evaluation period; generate a remote photoplethysmogram (PPG) signal based on the image data set; determine a recovered PPG signal by generating a frequency response based on a frequency-tuned filter bank and the remote PPG signal, the recovered PPG signal generated based on a peak magnitude of the frequency response; generate a predicted electrocardiogram (ECG) signal based on a prediction model including one or more prediction decoders tuned based on semantic features of the prior identified remote PPG signal; and determine, for display at a user device, user vitals data associated with the evaluation period.

One or more of the following features can be included in any feasible combination. For example, the frequency-tuned filter bank can be configured as a wavelet-based set of filters. The prediction model can include a plurality of prediction decoders respectively generating a predicted ECG signal based on the sole remote PPG signal, the plurality of predicted ECG signals representing ECG signals as if generated based on bioelectrode devices positioned on the user's body. The plurality of prediction decoders can generate a respective predicted ECG signal based on a subset of semantic data associated with the remote PPG signal.

The prediction model can include an encoder propagating semantic data sets associated with the remote PPG signal for downstream ECG signal prediction. The prediction model can comprise a single encoder and multiple decoder-based architecture. The image data set can include a video data stream representing a user's face over the evaluation period. The user vitals data can include health-related measurements associated with the user.

In another aspect, the present disclosure describes a method of detecting user vitals. The method may include receiving an image data set representing a user face over an evaluation period; generating a remote photoplethysmogram (PPG) signal based on the image data set; determining a recovered PPG signal by generating a frequency response based on a frequency-tuned wavelet-based filter-bank and the remote PPG signal, the recovered PPG signal generated based on a peak magnitude of the frequency response; generating a predicted electrocardiogram (ECG) signal based on a prediction model including one or more prediction decoders tuned based on semantic features of the prior identified remote PPG signal; and determining, for display at a user device, user vitals data associated with the evaluation period.

In another aspect, a non-transitory computer-readable medium or media having stored thereon machine interpretable instructions which, when executed by a processor may cause the processor to perform one or more methods described herein.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrates a set of waveforms showing a comparison of a remote PPG signal waveform and a reference PPG signal waveform, in accordance with embodiments of the present disclosure;

FIG. 2 illustrates a set of waveforms showing a comparison of a remote PPG signal waveform and an ECG signal waveform, in accordance with embodiments of the present disclosure;

FIG. 3 illustrates a system, in accordance with embodiments of the present disclosure;

FIG. 4 illustrates a magnitude of the frequency spectrum for the example remote PPG signal, in accordance with embodiments of the present disclosure;

FIG. 5 illustrates a representative frequency response of the continuous wavelet transform filter bank, in accordance with embodiments of the present disclosure;

FIG. 6 illustrates a magnitude response plot of a remote PPG signal using the filter bank, in accordance with embodiments of the present disclosure;

FIG. 7 illustrates signal waveforms associated with a remote PPG signal waveform, a recovered PPG signal waveform, and an ECG signal waveform, in accordance with embodiments of the present disclosure;

FIG. 8 illustrates a composite waveform showing a recovered PPG signal waveform superimposed on corresponding ECG signal waveform data, in accordance with embodiments of the present disclosure;

FIG. 9 illustrates a recovered remote PPG signal waveform superimposed on a corresponding ECG signal waveform following signal alignment operations, in accordance with embodiments of the present disclosure;

FIG. 10 illustrates a set of waveforms illustrating an array of signal waveforms for relative comparison, in accordance with embodiments of the present disclosure;

FIG. 11 illustrates a high-level block diagram of a 1D convolutional neutral network-based (CNN) encoder-decoder architecture for generating predicted ECG signal waveforms, in accordance with embodiments of the present disclosure;

FIG. 12 illustrates a block diagram showing details of a 1D CNN-based encoder-to-multiple decoder architecture for generating multiple ECG signal waveforms based on a sole input remote PPG signal waveform, in accordance with embodiments of the present disclosure;

FIG. 13 illustrates a set of signal waveforms showing twelve predicted ECG signal waveforms representing predicted data simulating acquisition of ECG data via twelve discrete bioelectrode devices positioned across a user's body, in accordance with embodiments of the present disclosure;

FIG. 14 illustrates a flowchart of a method for detecting user vitals data, in accordance with embodiments of the present disclosure;

FIG. 15 illustrates a flowchart of a method of training a model for remote PPG to ECG, in accordance with embodiments of the present disclosure; and

FIG. 16 illustrates a flowchart of a method of remote PPG to ECG generation, in accordance with embodiments of the present disclosure.

### DETAILED DESCRIPTION

Heath monitoring devices may be configured for monitoring user activity. For example, such devices may include operations for tracking user fitness data and measuring a user's physiological metrics, such as heart rate variability, glucose measures, blood pressure reading, or other health-related information.

In some examples, health monitoring devices may measure and track heart rate data. In some scenarios, electrocardiogram (ECG) may be used as a cardiac monitoring technique. ECGs may record electrical activity associated with a user's heart and may record variations of signal morphology over time. ECGs may be used to identify irregularities in heart rhythms, among other electrical characteristics associated with the user's heart.

ECG is a common method for assessing a user's cardiovascular health. ECG signals may represent electrical activity of a user's heart based on signals of two or more bioelectrodes positioned on a user's body. In some scenarios, bioelectrodes may be adhered to a user's skin and may cause discomfort when adhered to the user's skin for long durations of time. Further, bioelectrodes may be coupled to measurement devices via electrical wires thereby limiting user mobility.

Photoplethysmography (PPG) is an optically obtained plethysmogram used to detect blood volume changes in microvascular bed of tissue. PPG may be an optical measurement of volumetric changes in blood circulation. In some scenarios PPG may be used for determining heart rate statistics of a user.

As an example, PPG signals may include a pulsatile (AC) component and a superimposed (DC) component. The AC component may be associated with variations in blood volume that may arise from a user's heartbeat. The DC component may be shaped by user factors such as the user's respiration, sympathetic nervous system activity, or temperature regulation. The AC component may be associated with changes in blood volume corresponding to cardiac activity, such as systolic and diastolic phases.

In some scenarios, it may be desirable to provide systems and methods of detecting user vitals, including ECG measurements of user cardiac activity, based on non-contact devices and methods.

In some embodiments, systems and devices may be configured based on facial video-based remote physiological measurement methods for estimating remote PPG signals from video stream data. The video stream data may represent a series of images of a user's face over time. In some embodiments, systems and devices may then generate user vitals data, such as heart rate, respiration frequency, among other examples, based on the remote PPG signals. Features of embodiments of such systems, devices, and methods will be described in the present disclosure.

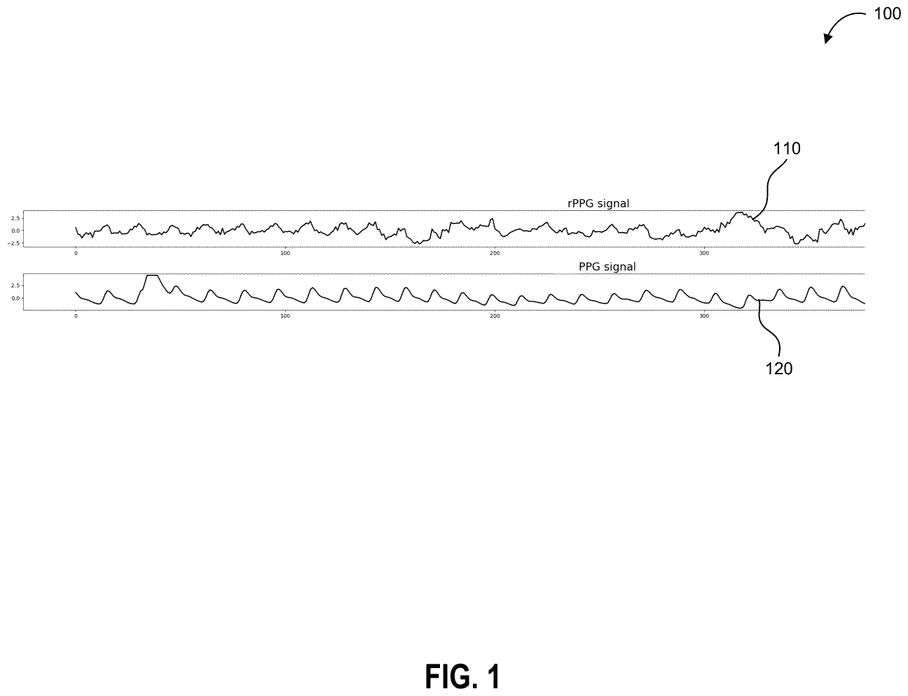

Reference is made to FIG. 1, which illustrates a set of waveforms 100 illustrating a comparison of a remote PPG signal waveform 110 and a PPG signal waveform 120, in accordance with embodiments of the present disclosure.

The remote PPG signal waveform 110 may be based on video stream data representing a user's face over time. The remote PPG signal waveform 110 may be generated based on the

video stream data using various methods (such as described in [13]), including blind source separation (BSS) algorithms which may be referred to as plane-orthogonal-to-skin (POS) algorithm. In some scenarios, remote PPG signal waveforms may be generated based on subtle color changes in facial skin regions of a user based on changes in blood volume in microvascular bed of tissue of a user.

The PPG signal waveform 120 may be a reference signal and may be generated based on touch-based sensors donned by a user. In some scenarios, the example PPG signal waveform 120 may be considered a ground-truth PPG, as the signal waveform may have been generated based on known touch-based sensors configured to be donned by the user.

In the example shown in FIG. 1, remote PPG signal waveforms 110 may include relatively noisy signal characteristics when compared to PPG signal waveforms 120.

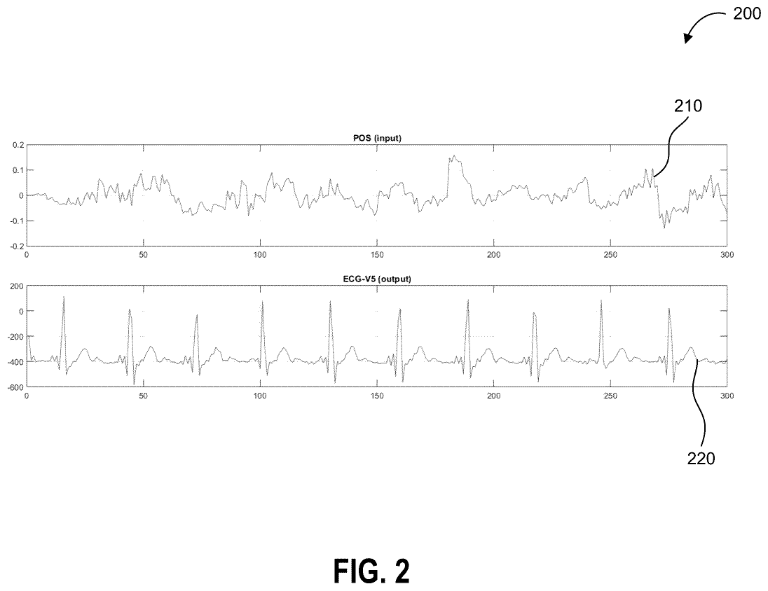

Reference is made to FIG. 2, which illustrates a set of waveforms 200 illustrating a comparison of a remote PPG signal waveform 210 and an ECG signal waveform 220, in accordance with embodiments of the present disclosure.

As described with reference to FIG. 1, remote PPG signal waveforms may include relatively noisy signal characteristics as compared to reference PPG signal waveforms 120. While there may be correlation between variations in color changes in facial skin regions and a user's heartbeat, remote PPG signal waveforms may not on its own be a suitable approximation for vital sign measurements as well as generating ECG signal waveforms.

In some examples, systems and methods have been developed to reconstruct ECG signal waveforms based on PPG signal (not remote PPG) waveforms. Some example approaches of reconstructing ECG signal waveforms from PPG signal waveforms may be based on cycle-based approaches, where accurate alignment and cycle segmentation may be required. [see e.g., [1], [2], [6], [7], or [9]] In some other example approaches, the trained models are subject user subject specific. These models may not be generalizable for a general population and may be limited in terms of usability.

In some examples, devices may be configured to reconstruct an ECG signal waveform directly based on video stream data see [11], where the reconstructed ECG signal waveform represents signals generated as if it were associated with a single bioelectrode device. In the present example where devices may be configured to reconstruct the ECG signal waveform directly from video stream data, such operations may not generate an ECG waveform that corresponds to actual heartbeat or may be out of sync from real ECG waveforms.

In some scenarios, it may be desirable to provide devices and methods of detecting user vitals data based on remote PPG signal waveforms derived from video stream data representing a user's face.

As will be described with reference to some embodiments in the present disclosure, mobile computing devices having one or more image capture devices may be configured for generating video stream data. For example, mobile computing devices may include smartphone devices having a camera-device positioned and operable to capture a stream of images or video stream data of a user. The mobile computing device may be operated by a user to obtain video stream data or image data representing the user's face without assistance from any other users to obtain “selfie” image data. Based on such video stream data, in some embodiments, systems and devices described herein may be configured to: (i) generate remote PPG signals based on the video stream data; and (ii) generate or reconstruct ECG signal waveforms based on the remote PPG signals.

In some embodiments, the reconstructed ECG signal waveforms may represent user data as if it were acquired via a single bioelectrode device and an ECG measurement apparatus. In some scenarios, ECG is acquired with numerous electrodes, such as 3-lead to 12-lead set of electrodes. As will be described in the present disclosure, in some embodiments, systems and methods may be configured to reconstruct an array of ECG signal waveforms based on the video stream data, where the array of ECG signal waveforms may represent user data as if it were acquired via a plurality of leads coupled to an ECG measurement apparatus.

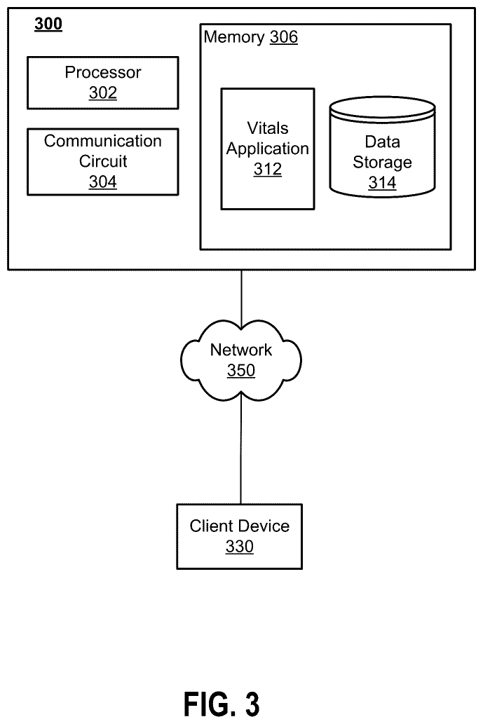

Reference is made to FIG. 3, which illustrates a system 300, in accordance with an embodiment of the present disclosure.

The system 300 may be configured to conduct operations of detecting user vitals based on video stream data. In some embodiments, detection of user vitals may be based on generated or reconstructed ECG signal waveforms. In some embodiments, generating the ECG signal waveforms may be based on remote PPG signal waveforms. Further, the remote PPG signal waveforms may be based on video stream data captured by a computing device associated with a user. For example, the computing device associated with the user may be a smartphone device, and the smartphone device may include an image capture device for generating video stream data of the user's face.

The system 300 may transmit or receive data messages via a network 350 to or from one or more client devices 330. A single client device 330 is illustrated in FIG. 3; however, it may be understood that any number of client devices may transmit or receive data messages to or from the system 300.

The client device 330 may be a computing device, such as a mobile device, a tablet device, a personal computer device, or a thin-client device that may include an image capture device. For example, the client device 330 may be a smartphone device having one or more image capture devices. The image capture devices may include a front-facing camera allowing a user to obtain “selfie” images or video stream data representing the user's face. Embodiments of methods described herein may be based on generating remote PPG signal waveforms representing detected blood volume changes in microvascular bed of tissue, which may be visually manifested based on subtle colour changes in facial skin regions of the user. Other example features of detecting physiological changes to a user's features may be contemplated.

The client device 330 may be configured to operate with the system 300 for executing data processes for generating ECG signal waveforms based on remote PPG signal waveforms derived from video stream data.

The client device 330 may include a processor, a memory, or a communication interface. In some embodiments, the client device 330 may be a computing device associated with a local area network. The client device 330 may be connected to the local area network and may transmit one or more data sets to the system 300.

The network 350 may include a wired or wireless wide area network (WAN), local area network (LAN), a combination thereof, or other networks for carrying telecommunication signals. In some embodiments, network communications may be based on HTTP post requests or TCP connections. Other network communication operations or protocols may be contemplated.

The system 300 includes a processor 302 configured to implement processor-readable instructions that, when executed, configure the processor 302 to conduct operations described in the present disclosure. For example, the system 300 may be configured to receive a plurality of image data streams or video data streams representing a user's face and may be configured to generate remote PPG signal waveforms. Remote PPG signal waveforms may be the basis for operations to construct ECG signal waveforms for deducing vitals data associated with the user.

In some examples, the processor 302 may be a microprocessor or microcontroller, a digital signal processing processor, an integrated circuit, a field programmable gate array, a reconfigurable processor, or combinations thereof.

The system 300 includes a communication circuit 304 configured to transmit or receive data messages to or from other computing devices, to access or connect to network resources, or to perform other computing applications by connecting to a network (or multiple networks) capable of carrying data.

In some embodiments, the network 350 may include the Internet, Ethernet, plain old telephone service line, public switch telephone network, integrated services digital network, digital subscriber line, coaxial cable, fiber optics, satellite, mobile, wireless, SS7 signaling network, fixed line, local area network, wide area network, or other networks, including one or more combination of the networks. In some examples, the communication circuit 304 may include one or more busses, interconnects, wires, circuits, or other types of communication circuits. The communication circuit 304 may provide an interface for communicating data between components of a single device or circuit.

The system 300 includes memory 306. The memory 306 may include one or a combination of computer memory, such as random-access memory, read-only memory, electro-optical memory, magneto-optical memory, erasable programmable read-only memory, and electrically-erasable programmable read-only memory, ferroelectric random-access memory, or the like. In some embodiments, the memory 306 may be storage media, such as hard disk drives, solid state drives, optical drives, or other types of memory.

The memory 306 may store a vitals application 312 including processor-readable instructions for detecting user vitals data based on video stream data.

In some examples, the vitals application 312 may include operations for retrieving video stream data received from one or more client devices 330 and generating remote PPG signal waveforms for downstream analysis. In some embodiments, generating remote PPG signal waveforms from retrieved video stream data may be based on blind source separation operations, among other example operations.

The vitals application 312 may include operations for generating an ECG signal waveform based on the constructed remote PPG signal waveform. Features of such operations will be described in the present disclosure.

In some embodiments, the vitals application 312 may generate two or more ECG signal waveforms based on the constructed remote PPG signal waveform. The two or more ECG signal waveforms may represent multiple ECG signals that correspond to signals that may have been obtained using respective bioelectrode devices if the ECG signal had been generated using bioelectrode devices affixed to a user. That is, the generated one or more ECG signal waveforms may provide a simulated generation of ECG signals that otherwise may be generated based on ECG lead outputs.

The system 300 includes data storage 314. In some embodiments, the data storage 314 may be a secure data store. In some embodiments, the data storage 314 may store training data sets for training models for generating remote PPG signal waveforms or ECG signal waveforms from video stream data. In some embodiments, the data store 314 may include ground truth PPG and ground truth ECG signal waveforms for correlating with test data sets for training models of the vitals application 312.

The data storage 314 may store video data streams received from one or a plurality of client devices 330 for downstream processing or generation of signal waveforms. Other types of data sets received from the client device 330 may be stored in the data storage 314.

As described herein, the system 300 may be configured to conduct operations for detecting user vitals data based on video stream data, thereby providing a non-contact generation of signal waveforms for deducing user vitals data. For example, a user may generate video stream data representing the user's face for a duration of time with a smartphone device. The vitals application 312 may include operations for constructing remote PPG signal waveforms, and subsequently ECG signal waveforms based on the video stream data for deducing user vitals data.

In some embodiments, the system 330 may conduct operations for generating remote PPG signal waveforms from video stream data based on blind source separation algorithm.

Referring again to FIG. 1, remote PPG signals 110 may be relatively noisy as compared to PPG signals generated based on user touch-based sensors. In some examples, this may require filtering and recovery of remote PPG signals described with reference to figures of the present disclosure.

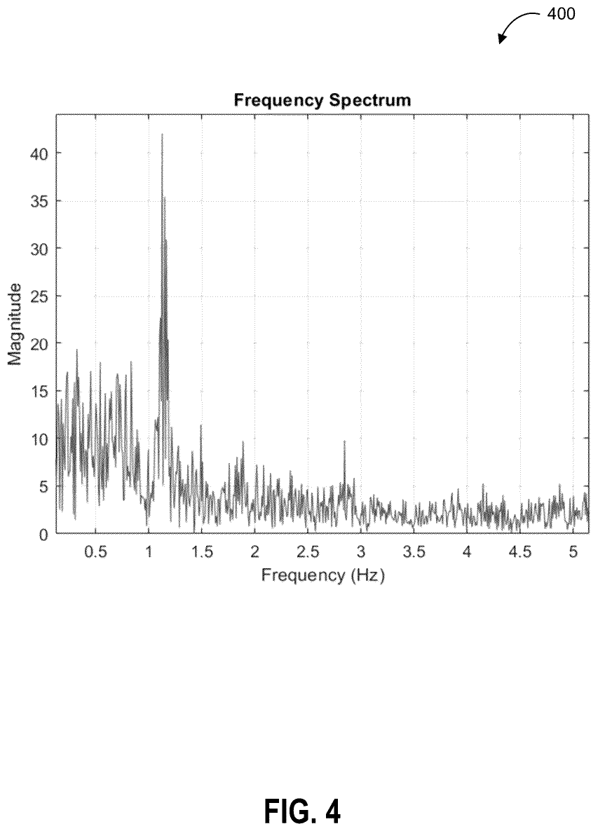

Reference is made to FIG. 4, which illustrates a frequency spectrum of remote PPG signal 400, in accordance with an embodiment of the present disclosure. The frequency spectrum 400 representing the remote PPG signal waveform illustrates a dominant peak in combination with a plurality of smaller peaks corresponding to harmonic and noise components.

To generate a suitably representative PPG signal waveform based on the remote PPG signal waveform, the vitals application 312 may include filtering of remote PPG with wavelet-based, narrow-band, filter-bank operations. For instance, operations of wavelet-based analysis allow signal to be decomposed both in frequency and time domains.

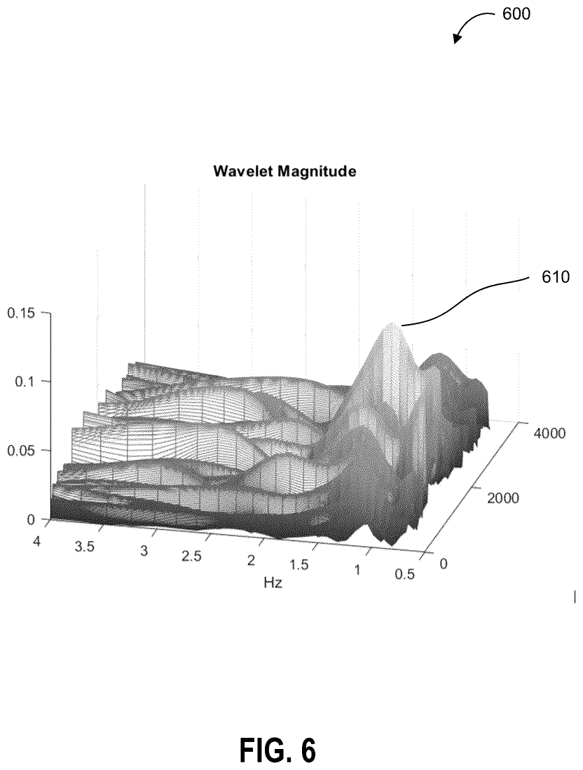

In some embodiments, the vitals application 312 may include operations to filter the remote PPG signal waveform data with a set of narrow-band filters in the filter-bank, and subsequently selecting a filter response corresponding to the maximum magnitude among all of the filter responses (shown in FIG. 6). If n is the number of filters and m is the signal length then after wavelet transform a 2-dimensional complex array W_{i,j} of size [m, n] is generated. The index of the filter corresponding to the maximum response is estimated as:

$$I = argmax ( {\sum }_{i = 1}^{m} \vert {W}_{ij} \vert ) , j = 1 , 2 , … n$$

where operator |·| indicates magnitude of the complex array. The recovered PPG signal is simply, y=R(Wᵤ), for all i=1,2, . . . m, where operator R indicates the real part of the complex numbers.

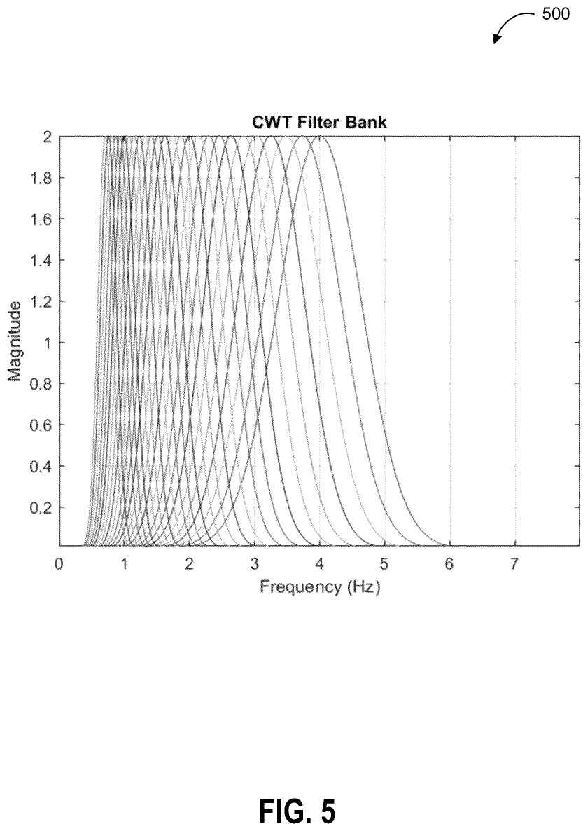

Reference is made to FIG. 5, which illustrates a representative frequency plot 500 of a continuous wavelet transform filter bank, in accordance with an embodiment of the present disclosure. The frequency plot 500 illustrates the magnitude of respective filters in an array of filters having center frequencies.

FIG. 6 illustrates a magnitude response plot 600 of a remote PPG signal waveform based on the example continuous wavelet transform filter bank, in accordance with embodiments of the present disclosure. For example, the magnitude response plot 600 may be associated with a remote PPG signal waveform following operations of the example continuous wavelet transform filter bank associated with the representative frequency plot 500 of FIG. 5.

In some embodiments, the vitals application 312 may conduct operations to determine a maximum magnitude identified on the magnitude response plot 600 of the frequency responses associated with the example continuous wavelet transform filter bank.

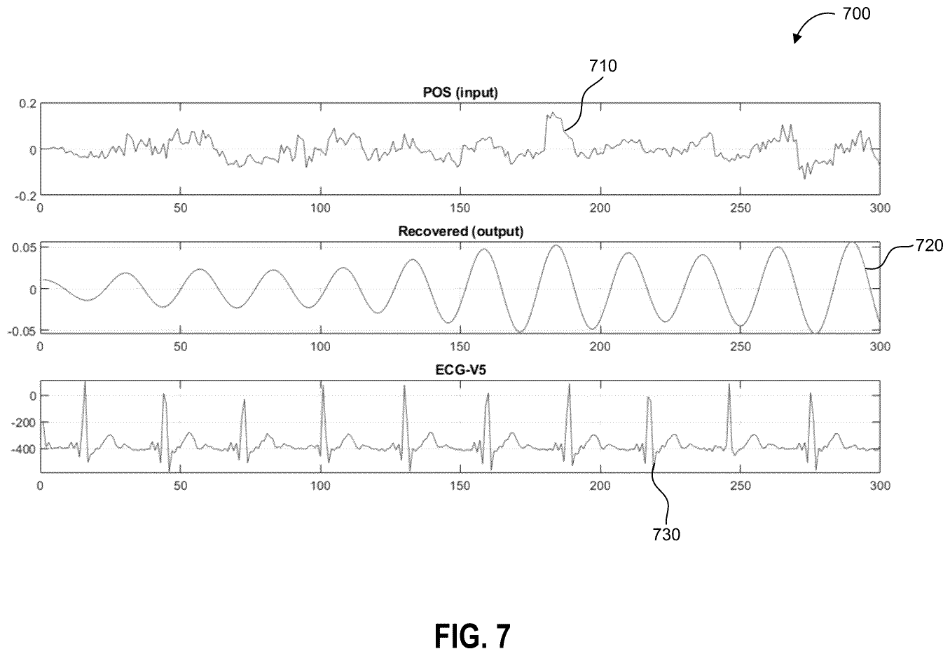

Reference is made to FIG. 7, which illustrates signal waveforms 700 associated with a remote PPG signal waveform 710, a recovered PPG signal waveform 720, and an ECG signal waveform 730, in accordance with an embodiment of the present disclosure. The signal waveforms illustrated in FIG. 7 provide a comparative view of the array of signal waveforms.

The ECG signal waveform 730 may be considered a ground truth ECG signal waveform generated based on one or more electrodes affixed to a user whilst a video data stream is acquired representing a user's face.

In FIG. 7, the recovered PPG signal waveform 720 shows waveform features corresponding to a maximum amplitude among the filter frequency responses.

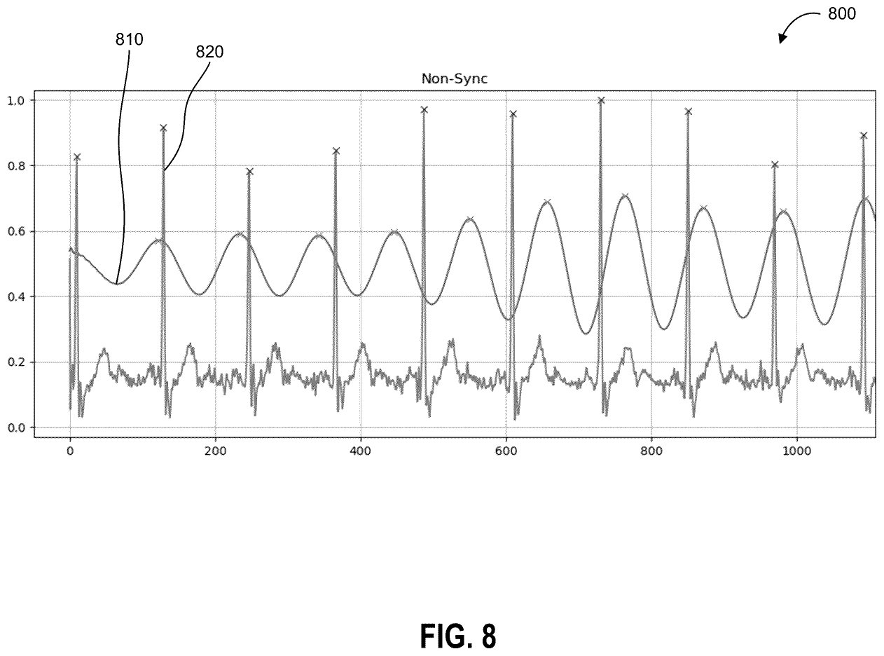

In some scenarios, remote PPG signal waveform data (corresponding to video stream data) and corresponding ECG signal waveform data collected (for comparison) in a clinical setting may not be synchronized in time. To illustrate, reference is made to FIG. 8, which illustrates a composite waveform 800 illustrating a recovered remote PPG signal waveform superimposed on corresponding ECG signal waveform. In FIG. 8, magnitude peaks may be mis-aligned in time.

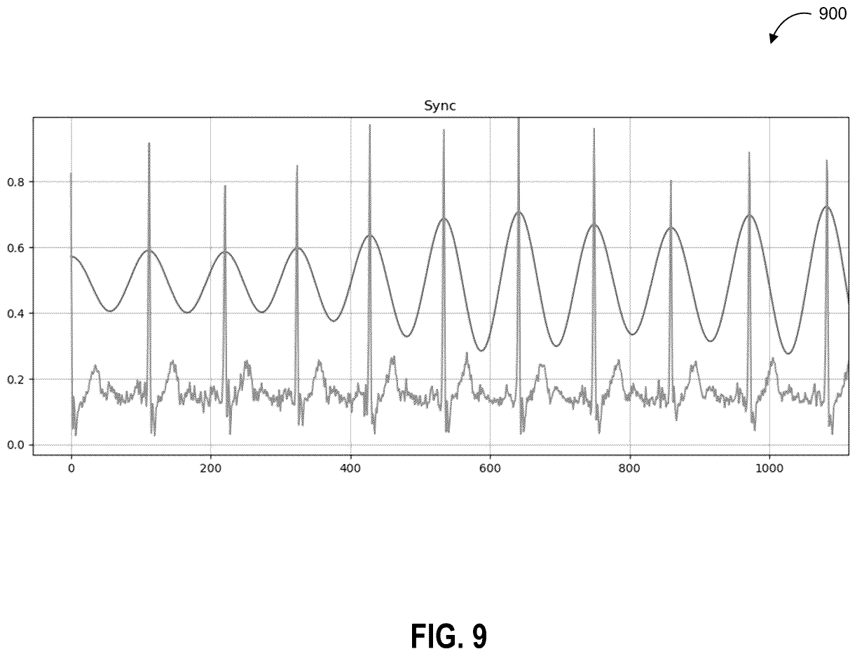

Thus, in some embodiments, the vitals application 312 may include operations of peak detection-based signal alignment operations for aligning the respective remote PPG signal waveform data with ECG signal waveform data. To illustrate, FIG. 9 shows a recovered remote PPG signal waveform superimposed on a corresponding ECG signal waveform following peak detection-based signal alignment operations, in accordance with embodiments of the present disclosure.

In some embodiments, the vitals application 312 may include operations of peak detection-based signal alignment operations during model training based on training data sets. In some scenarios, such operations of peak detection-based signal alignment operations may not be executed whilst doing predictions for deducing user vitals data based on video stream data.

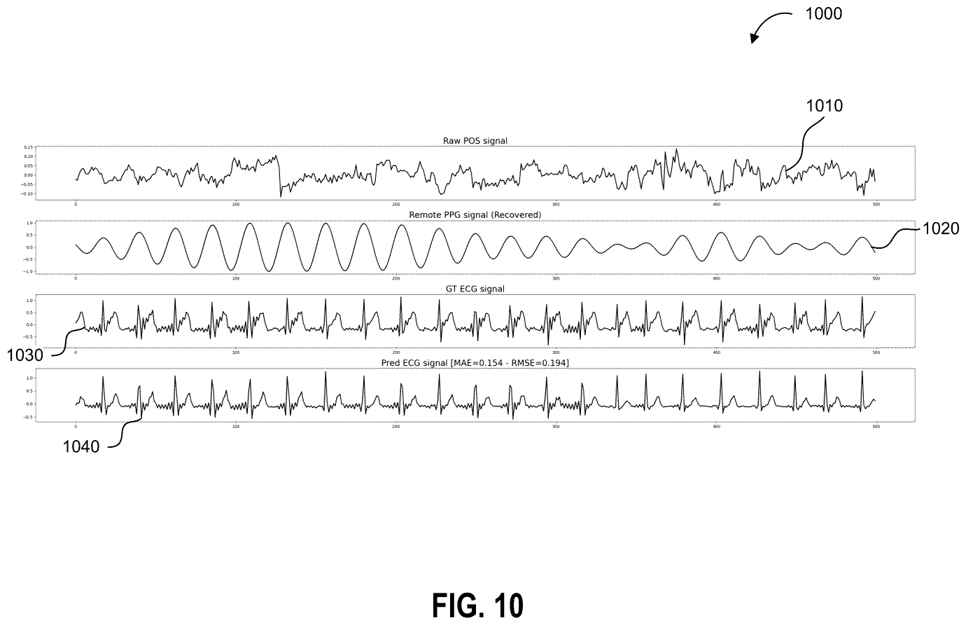

Reference is made to FIG. 10, which illustrates a set of waveforms 1000 illustrating an array of signal waveforms for relative comparison, in accordance with an embodiment of the present disclosure.

The set of waveforms 1000 include a remote PPG signal waveform 1010, a reconstructed PPG signal waveform 1020 based on operations of a wavelet-based filter bank framework, a ground truth ECG signal waveform 1030 for comparison, and a predicted ECG signal waveform 1040 based on the reconstructed PPG signal 1020.

The remote PPG signal waveform 1010 may have been constructed based on blind source separation operations and video stream data representing a user's face over time. The reconstructed PPG signal waveform 1020 may be based on operations of the wavelet-based filter bank framework described in the present disclosure. The ground truth ECG signal waveform 1030 may be a signal waveform generated based on one or more bioelectrode devices positioned on a user's body for detecting user data whilst the user may have been capturing video stream data of the user's face over time.

Further, the predicted ECG signal waveform 1040 may be generated based on the recovered PPG signal 1020 based on an encoder-decoder deep-learning framework provided in the present disclosure.

As illustrated in FIG. 10, the predicted ECG signal may be generated based on the reconstructed PPG signal waveform 1020 as an intermediary step from the extracted remote PPG signal waveform 1010.

The illustrated generation of the predicted ECG signal waveform 1040 may be based on operations leveraging a maximum magnitude of frequency responses associated with operations of the continuous wavelet transform filter bank framework applied to the remote PPG signal waveform 1010.

Accordingly, FIG. 10 illustrates the predicted ECG signal waveform 1040 as providing a corresponding signal waveform like a ground-truth ECG signal waveform 1030.

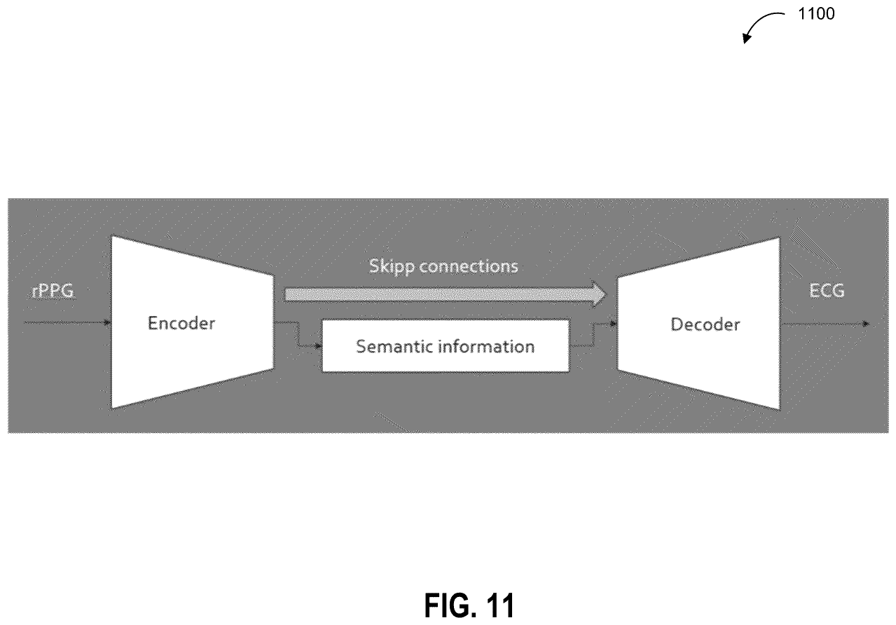

Reference is made to FIG. 11, which illustrates a high-level block diagram 1100 of a 1D CNN-based encoder-decoder architecture for generating predicted ECG signal waveforms based on remote PPG signal waveforms, in accordance with embodiments of the present disclosure.

The block diagram 1100 illustrated in FIG. 11 provides a predicted ECG signal representing ECG signal data as if it were associated with a single bioelectrode device affixed to a user during ECG data acquisition.

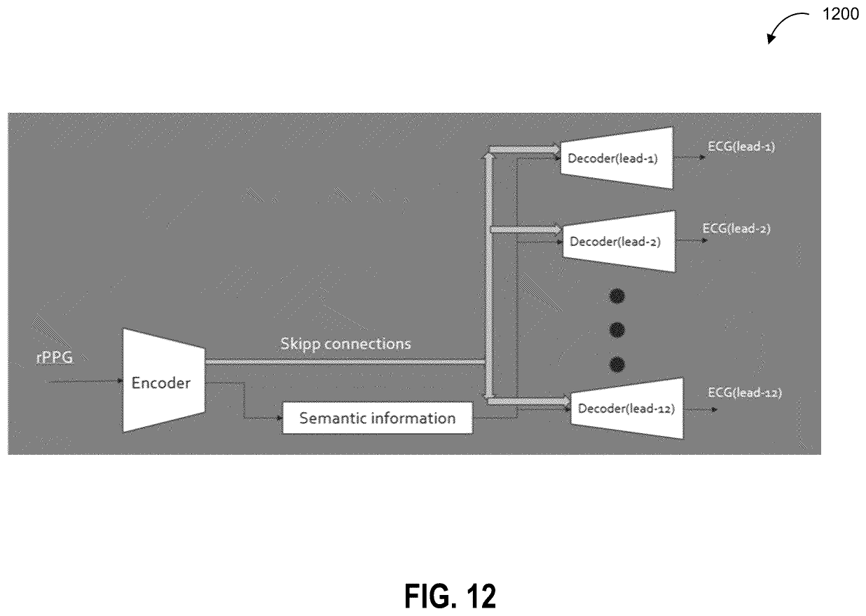

Reference is made to FIG. 12, which illustrates a block diagram 1200 illustrating details of a 1D CNN-based encoder-to-multiple decoder architecture for generating multiple ECG signal waveforms based on a sole input remote PPG signal waveform, in accordance with embodiments of the present disclosure.

In some embodiments, the encoder-to-multiple decoder architecture for generating multiple ECG signal waveforms may include operations for propagating signal semantic data and SKIPP connections from the encoder to plurality of decoders.

In scenarios were operations of the encoder block may generate signals representing semantic features of waveforms potentially representing signals as if it were generated by a plurality of bioelectrodes for hardwired ECG signal waveform generation, embodiments of the present disclosure may include a plurality of decoder blocks for generating predicted ECG signal waveforms corresponding to representations as if the signals were generated by the plurality of bioelectrodes for hardwired ECG signal waveform generation.

Accordingly, in some embodiments, a plurality of decoders (e.g., 12 decoders) may be respectively provided for generating signal waveforms representing signals as if the signals were generated by the plurality of bioelectrodes for hardwired ECG signal waveform generation. In some embodiments, the respective decoders may be configured to generate a predicted ECG signal waveform for particular characteristics as if the signal were generated by a bioelectrode positioned at an upper left portion of a user's chest, a lower left portion of the user's chest, an upper right portion of the user's chest, among other example positions of notional bioelectrode devices.

In some embodiments, the plurality of decoders may be configured to generate a predicted ECG signal waveform as if it were a signal generated by a particular electrode positioned at a desired position on the user's body. Such decoders may have been trained based on training data sets having particular ECG data corresponding to discrete bioelectrode device positioning on the user's body.

For example, respective decoders representing bioelectrode leads 1 to 12 may respectively have been trained on training data sets providing semantic data for associating with bioelectrodes that may be positioned on a user's chest, on a user's back, on a user's arm, or other portions of the user's body. As an example, subsets of semantic data may be propagated to decoders based on the category of anticipated bioelectrode device placement (e.g., inferior ECG leads, lateral ECG leads, septal ECG leads, or anterior ECG leads).

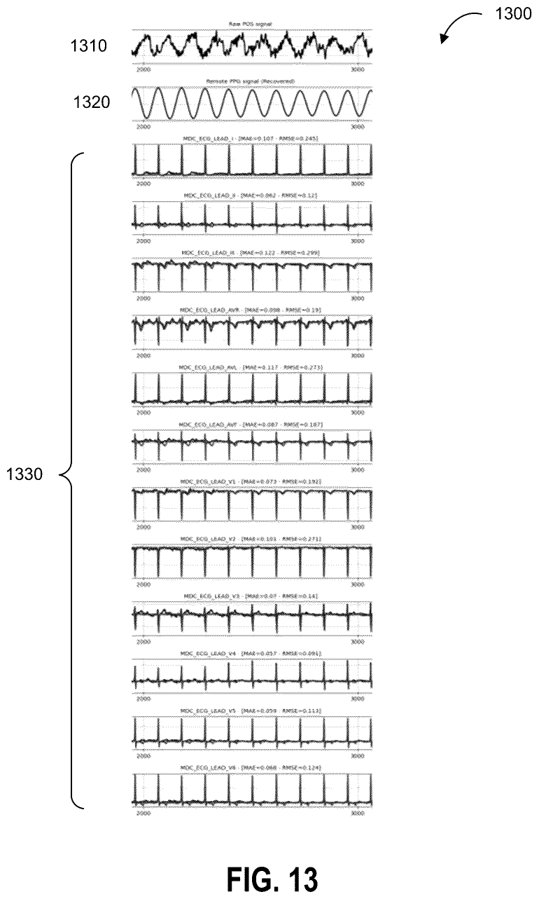

Reference is made to FIG. 13, which illustrates a set of signal waveforms 1300 illustrating 12 predicted ECG signal waveforms representing predicted data simulating acquisition of ECG data via 12 discrete bioelectrode devices positioned across a user's body, in accordance with embodiments of the present disclosure.

The set of signal waveforms 1300 shows an illustration of a remote PPG signal waveform 1310 generated based on video stream data representing a user's face over time. In some embodiments, the video stream data may represent approximately 60 seconds of video footage of the user's face for capturing subtle physiological changes of the user's face that may represent user vitals data. In some embodiments, the remote PPG signal waveform 1310 may be generated based on blind source separations in combination with other unsupervised operations.

The set of signal waveforms 1300 may include a recovered PPG signal waveform 1320. The recovered PPG signal waveform 1320 may be generated based on embodiments of continuous wavelet transform filter bank operations described in the present disclosure. In some scenarios, the continuous wavelet transform filter bank operations may be configured for ameliorating undesired noise artifacts and harmonics of the extracted remote PPG signal waveform.

The set of signal waveforms 1300 illustrated in FIG. 13 show twelve discrete predicted ECG signal waveforms respectively representing signals that are predicted to have been acquired if discrete bioelectrode devices were positioned across a user's body via ECG leads for generating ECG waveforms.

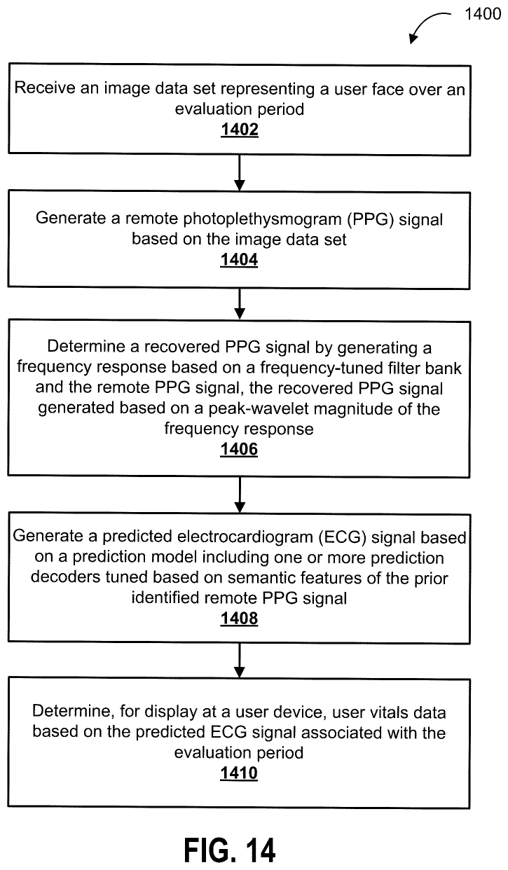

Reference is made to FIG. 14, which illustrates a flowchart of method 1400 for detecting user vitals, in accordance with embodiments of the present disclosure. The method may be conducted by the processor 302 of the system 300 (FIG. 3). Processor-readable instructions may be stored in the memory 306 and may be associated with the vitals application 312 or other processor readable applications not illustrated in FIG. 3. The method 1400 may include operations, such as data retrievals, data manipulations, data storage, or the like, and may include other computer executable functions.

In some scenarios, a user may be operating the client device 330 (FIG. 3) whilst conducting exercise routines or whilst conducting operations for tracking their own health. In some embodiments, the user may be operating the client device 330 while assessing the user vitals in a resting state. In some embodiments, the client device 330 may include one or more image capture devices for generating video stream data. In some examples, the image capture device may be positioned such that the user may capture “selfie image” or “selfie video” content of the user's face.

In scenarios where a user desires to detect user vitals data, such as health or fitness tracking data, the user may operate the client device 330 for obtaining video stream data representing the user's face for 60 seconds or another duration of time. In some scenarios, the video stream data representing the user's face may capture subtle physiological changes of the user's face that may be useful for deducing user vitals data. For example, blood volume changes in microvascular bed of tissue may be correlated with subtle colour changes in facial skin regions of a user.

At operation 1402, the processor receives an image data set representing a user face over an evaluation period. In some embodiments, the image data set may be video stream data of a user's face for 60 seconds and acquired by a front facing camera of a smartphone device (e.g., client device 330-FIG. 3).

In some scenarios, an application may prompt the user to capture a video clip of the user's face for the purpose of detecting user vitals data. The user may utilize the client device 330 for capturing the video stream data and the client device 330 may transmit the video stream data to the system 300. The processor 302 (FIG. 3) may then receive the video stream data representing the user's face for the evaluation period.

At operation 1404, the processor may generate a remote PPG signal based on the image data set. In some embodiments, the processor may conduct blind source separation operations for generating the remote PPG signal based on the image data set. In some embodiments, the processor may conduct operations of blind source separation in combination with one or more operations for physiological signal recovery.

At operation 1406, the processor may determine a recovered PPG signal by generating a frequency response based on a frequency-tuned wavelet-based filter-bank and the remote PPG signal. The recovered PPG signal may be generated based on a peak wavelet magnitude of the frequency response. In some embodiments, the frequency-tuned filter bank may be configured as a wavelet-based set of filters.

For example, the processor may conduct operations to determine a maximum magnitude identified on a magnitude response plot 600 (FIG. 6) of the frequency responses associated with a continuous wavelet transform filter-bank and generate the recovered PPG signal.

At operation 1408, the processor may generate a predicted ECG signal based on a prediction model including one or more prediction decoders tuned based on semantic features of the prior generated remote PPG signal.

In some embodiments, the prediction model may include an encoder propagating semantic data sets associated with the generated remote PPG signal for downstream ECG signal prediction.

In some embodiments, the prediction model may include a plurality of prediction decoders respectively generating a predicted ECG signal from the sole remote PPG signal. The plurality of predicted ECG signals may represent ECG signals that would be generated if generated based on bioelectrode devices positioned on the user's body.

In some embodiments, the plurality of prediction decoders may generate a respective predicted ECG signal based on a subset of semantic data of the remote PPG signal. For example, the semantic data propagated to respective prediction decoders may be based on the type of anticipated bioelectrode device placement position. That is, as inferior ECG leads, lateral ECG leads, septal ECG leads, or anterior ECG leads are positioned at slightly varied positions of the user's body, respective prediction decoders may receive a subset of semantic data based on the type of ECG lead being simulated.

At operation 1410, the processor may determine, for display at a user device, user vitals data based on the predicted ECG signal associated with the evaluation period. In some embodiments, the processor may deduce heart health statistics associated with the user based on the video stream data for the evaluation period. Deduced heart health statistics may include heart rate, heart rate variability, heart rhythm, or other cardiac-related data for cardiac health diagnosis.

Embodiments described in the present disclosure are directed to predicting ECG data based on remote PPG signals generated from video stream data. In some other embodiments, based on video stream data representing the user's face for a duration of time, the system 300 may be configured to generate other types of prediction data sets for deducing user vitals data.

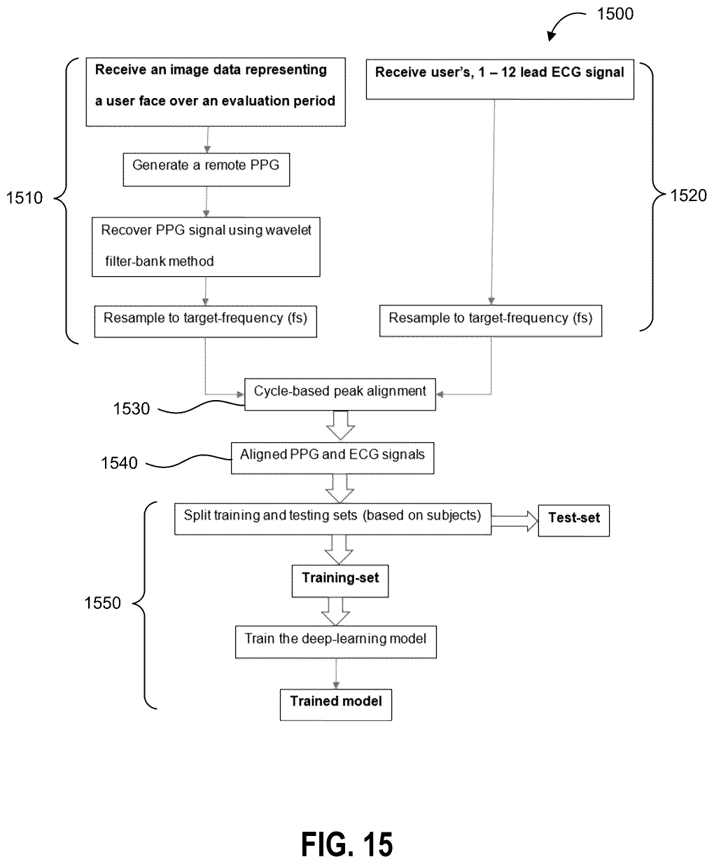

Reference is made to FIG. 15, which illustrates a flowchart of a method 1500 of training a model for detecting user vitals, in accordance with embodiments of the present disclosure. The method may be conducted by the processor 302 of the system 300 (FIG. 3).

A first series of operations 1510 may include receiving an image data representing a user face over an evaluation period. The operations may include generating a remote PPG signal. The operations may include generating a recovered PPG signal based on a wavelet-based filter-bank method. The operations may include resampling the generated signal to resample to a target frequency (fs).

A second series of operations 1520 may include receiving ECG signals of a user based on signals detected from ECG leads. In some embodiments, the ECG signals may be acquired based on 1 to 12 leads. The operations may include resampling the ECG signals to a target frequency (fs).

At operation 1530, a processor may conduct cycle-based peak alignment operations.

At operation 1540, a processor may conduct operations to align PPG and ECG signals.

A third series of operations 1530 may include operations for splitting training and testing sets based on users or subjects to derive test data sets and training data sets.

The operations may include training a deep-learning model for providing a trained model for detecting user vitals. The trained model for detecting user vitals may be part of the vitals application 312 or other processor readable applications of the system 300.

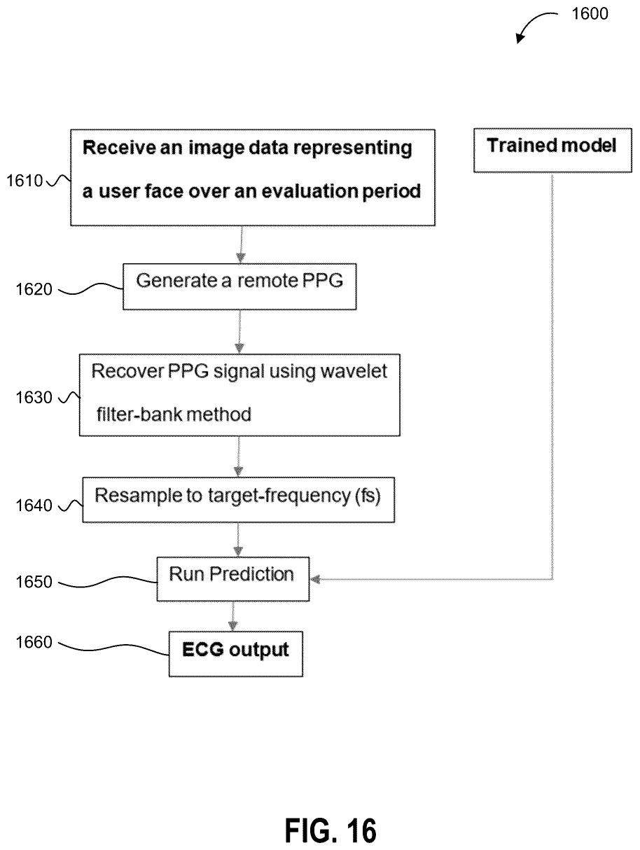

Reference is made to FIG. 16, which illustrates a flowchart of a method 1600 for detecting user vitals, in accordance with an embodiment of the present disclosure. The method may be conducted by the processor 302 of the system 300 (FIG. 3).

At operation 1610, the processor may receive image data representing a user face over an evaluation period. In some embodiments, the image data set may be video stream data of a user's face for 60 seconds and acquired by a front facing camera of a smartphone device (e.g., client device 330-FIG. 3).

At operation 1620, the processor may generate a remote PPG signal based on the image data set.

At operation 1630, the processor may generate a recovered PPG signal based on a wavelet filter-bank method described in the present disclosure.

At operation 1640, the processor may resample the recovered PPG signal to a target-frequency (fs).

At operation 1650, the processor may generate a prediction based on a trained model. The prediction may be a prediction of user vitals or user health-metric measurements. The trained model may be the trained model for detecting user vitals. In some embodiments, the prediction may be a predicted ECG signal based on the recovered PPG signal. The predicted ECG signal may correspond to an ECG signal associated with a user if ECG leads had been affixed to the user.

At operations 1660, the processor may display the predicted ECG signal representing a prediction of an ECG signal that would have been generated of a user if ECG leads had been affixed to the user.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes several instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

### REFERENCES

[1] Q. Zhu, X. Tian, C. W. Wong and M. Wu, “ECG Reconstruction via PPG: A Pilot Study,” 2019 IEEE EMBS International Conference on Biomedical & Health Informatics (BHI), Chicago, IL, USA, 2019, pp. 1-4, doi: 10.1109/BHI.2019.8834612.
[2] X. Tian, Q. Zhu, Y. Li and M. Wu, “Cross-Domain Joint Dictionary Learning for ECG Reconstruction from PPG,” ICASSP 2020-2020 IEEE International Conference on Acoustics, Speech and Signal Processing (ICASSP), Barcelona, Spain, 2020, pp. 936-940, doi: 10.1109/ICASSP40776.2020.9054242.
[3] H.-Y. Chiu, H.-H. Shuai and P. C. P. Chao, “Reconstructing QRS Complex from PPG by Transformed Attentional Neural Networks,” in IEEE Sensors Journal, vol. 20, no. 20, pp. 12374-12383, 15 Oct., 2020, doi: 10.1109/JSEN.2020.3000344.
[4] K. Vo, E. K. Naeini, A. Naderi, D. Jilani, A. M. Rahmani, N. Dutt, and H. Cao. P2E-WGAN: ECG waveform synthesis from PPG with conditional wasserstein generative adversarial networks. In Proceedings of the 36th Annual ACM Symposium on Applied Computing (SAC '21). ACM, New York, NY, USA, 2021, 1030-1036.
[5] Pritam Sarkar and Ali Etemad, CardioGAN: Attentive Generative Adversarial Network with Dual Discriminators for Synthesis of ECG from PPG, 2020, arXiv 2020.00104.
[6] Q. Zhu, X. Tian, C. W. Wong and M. Wu, “Learning Your Heart Actions From Pulse: ECG Waveform Reconstruction From PPG,” in IEEE Internet of Things Journal, vol. 8, no. 23, pp. 16734-16748, 1 Dec., 2021, doi: 10.1109/JIOT.2021.3097946.
[7] Omer, O.A., Salah, M., Hassan, A. M., Mubarak, A. S. (2022). Beat-by-beat ECG monitoring from photoplythmography based on scattering wavelet transform. Traitement du Signal, Vol. 39, No. 5, pp. 1483-1488. https://doi.org/10.18280/ts.390504.
[8] Tang Q, Chen Z, Guo Y, Liang Y, Ward R, Menon C, Elgendi M. Robust Reconstruction of Electrocardiogram Using Photoplethysmography: A Subject-Based Model. Front Physiol. 2022, Apr. 25; 13:859763. doi: 10.3389/fphys.2022.859763. PMID: 35547575; PMCID: PMC9082149.
[9] Abdelgaber K M, Salah M, Omer O A, Farghal A E A, Mubarak A S. Subject-Independent per Beat PPG to Single-Lead ECG Mapping. Information. 2023; 14(7):377. https://doi.org/10.3390/info14070377.
[10] Tang Q, Chen Z, Ward R, Menon C, Elgendi M. PPG2ECGps: An End-to-End Subject-Specific Deep Neural Network Model for Electrocardiogram Reconstruction from Photoplethysmography Signals without Pulse Arrival Time Adjustments. Bioengineering. 2023; 10(6): 630.
[11] Bin Li, Wei Zhang, Xiaobai Li, Hong Fu, Feng Xu, ECG signal reconstruction based on facial videos via combined explicit and implicit supervision, Knowledge-Based Systems, Elsevier, 2023.
[12] Wenjin Wang, Bert den Brinker, Sander Stuijk, and Gerard de Haan, “Algorithmic Principles of Remote-PPG”, IEEE TRANSACTIONS ON BIOMEDICAL ENGINEERING, VOL. PP, NO. 99, MONTH 2016.
[13] Haugg, Fridolin, Mohamed Elgendi, and Carlo Menon. “Effectiveness of Remote PPG Construction Methods: A Preliminary Analysis” Bioengineering 9, no. 10:485. https://doi.org/10.3390/bioengineering9100485, 2022.

What is claimed is:

1. A system for detecting user vitals comprising:
a processor;
a memory coupled to the processor and storing processor-executable instructions that, when executed, configure the processor to:
receive an image data set representing a user face over an evaluation period;
generate a remote photoplethysmogram (PPG) signal based on the image data set;
determine a recovered PPG signal by generating a frequency response based on a frequency-tuned filter bank and the remote PPG signal, the recovered PPG signal generated based on a peak magnitude of the frequency response;
generate a predicted electrocardiogram (ECG) signal based on a prediction model including one or more prediction decoders tuned based on semantic features of the prior identified remote PPG signal; and
determine, for display at a user device, user vitals data associated with the evaluation period.

2. The system of claim 1, wherein the frequency-tuned filter bank is configured as a wavelet-based set of filters.

3. The system of claim 1, wherein the prediction model includes a plurality of prediction decoders respectively generating a predicted ECG signal based on the sole remote PPG signal, the plurality of predicted ECG signals representing ECG signals as if generated based on bioelectrode devices positioned on the user's body.

4. The system of claim 3, wherein the plurality of prediction decoders generate a respective predicted ECG signal based on a subset of semantic data associated with the remote PPG signal.

5. The system of claim 1, wherein the prediction model includes an encoder propagating semantic data sets associated with the remote PPG signal for downstream ECG signal prediction.

6. The system of claim 1, wherein the prediction model comprises a single encoder and multiple decoder-based architecture.

7. The system of claim 1, wherein the image data set includes a video data stream representing a user's face over the evaluation period.

8. The system of claim 1, wherein the user vitals data includes health-related measurements associated with the user.

9. A method of detecting user vitals comprising:
receiving an image data set representing a user face over an evaluation period;
generating a remote photoplethysmogram (PPG) signal based on the image data set;
determining a recovered PPG signal by generating a frequency response based on a frequency-tuned filter bank and the remote PPG signal, the recovered PPG signal generated based on a peak magnitude of the frequency response;
generating a predicted electrocardiogram (ECG) signal based on a prediction model including one or more prediction decoders tuned based on semantic features of the prior identified remote PPG signal; and
determining, for display at a user device, user vitals data associated with the evaluation period.

10. The method of claim 9, wherein the frequency-tuned filter bank is configured as a wavelet-based set of filters.

11. The method of claim 9, wherein the prediction model includes a plurality of prediction decoders respectively generating a predicted ECG signal based on the sole remote PPG signal, the plurality of predicted ECG signals representing ECG signals as if generated based on bioelectrode devices positioned on the user's body.

12. The method of claim 11, wherein the plurality of prediction decoders generate a respective predicted ECG signal based on a subset of semantic data associated with the remote PPG signal.

13. The method of claim 9, wherein the prediction model includes an encoder propagating semantic data sets associated with the remote PPG signal for downstream ECG signal prediction.

14. The method of claim 9, wherein the prediction model comprises a single encoder and multiple decoder-based architecture.

15. The method of claim 9, wherein the image data set includes a video data stream representing a user's face over the evaluation period.

16. The method of claim 9, wherein the user vitals data includes health-related measurements associated with the user.

17. A non-transitory computer-readable medium having stored thereon machine interpretable instructions which, when executed by a processor, cause the processor to perform a computer implemented method of detecting user vitals comprising:
receiving an image data set representing a user face over an evaluation period;
generating a remote photoplethysmogram (PPG) signal based on the image data set;
determining a recovered PPG signal by generating a frequency response based on a frequency-tuned filter bank and the remote PPG signal, the recovered PPG signal generated based on a peak magnitude of the frequency response;
generating a predicted electrocardiogram (ECG) signal based on a prediction model including one or more prediction decoders tuned based on semantic features of the prior identified remote PPG signal; and
determining, for display at a user device, user vitals data associated with the evaluation period.

18. The non-transitory computer-readable medium of claim 17, wherein the frequency-tuned filter bank is configured as a wavelet-based set of filters.

19. The non-transitory computer-readable medium of claim 17, wherein the prediction model includes a plurality of prediction decoders respectively generating a predicted ECG signal based on the sole remote PPG signal, the plurality of predicted ECG signals representing ECG signals as if generated based on bioelectrode devices positioned on the user's body.

20. The non-transitory computer-readable medium of claim 19, wherein the plurality of prediction decoders generate a respective predicted ECG signal based on a subset of semantic data associated with the remote PPG signal.
